(19) United States
(12) Patent Application Publication
(10) Pub. No.: US 2004/0230283 A1
(43) Pub. Date: Nov. 18, 2004

(54) Trans-septal pacing method and apparatus

(75) Inventors: Frits W. Prinzen, Maastricht (NL); Chester L. Struble, Eijsden (NL)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/832,738

(22) Filed: Apr. 27, 2004

Related U.S. Application Data
(63) Continuation-in-part of application No. 10/360,765, filed on Nov. 29, 2001, now abandoned.

(51) Int. Cl. A61N 1/05

(52) U.S. Cl. 607/126

(57) ABSTRACT

A medical electrical trans-septal pacing lead includes a lead body, a tine-like structure terminating a distal end of the lead body and a distal electrode coupled to the lead body at a position proximal to and in close proximity to the structure. A method for delivering left ventricular pacing to a heart includes inserting the trans-septal pacing lead through an inter-ventricular septal wall of the heart, from a right ventricle to a left ventricle, and positioning the distal electrode in a left ventricular endocardial surface of the septal wall.

### RELATED APPLICATIONS

This application is a continuation-in-part of U.S. Application 60/333,762, which is incorporated by reference in its entirety herein; U.S. Application 60/333,762 was filed Nov. 29, 2001 and converted from a provisional to a non-provisional application on Nov. 29. 2002, under Ser. No. 10/360,765. Furthermore, cross-reference is hereby made to the commonly assigned related U.S. application Ser. No. XX/XXX,XXX (Attorney Docket No. P-9774.07) entitled “Papillary Muscle Stimulation” filed concurrently herewith and incorporated by reference in its entirety herein.

### TECHNICAL FIELD

The present invention relates to implantable medical devices and more particularly to pacing via a trans-septal approach.

### BACKGROUND

Patients with poor atrio-ventricular conduction or poor sinus node function typically receive pacemaker implants to restore a normal heart rate. For another set of patients suffering from left bundle branch block (LBBB), left ventricular pacing and/or bi-ventricular pacing has been shown to significantly improve cardiac hemodynamics and quality of life. However, some studies have shown that traditional pacing from a right ventricular (RV) apex can impair cardiac pumping performance. In some instances, ventricular wall abnormalities (ventricular remodeling) resulting from RV apical pacing have also been observed. So, alternative sites have been found where pacing can cause an electrical activation sequence similar to that in a normally activated heart and thus contribute to improved cardiac pump function.

From the literature there appear to be three major characteristics of normal cardiac electrical activation: 1.) Earlier activation of the left ventricle than right ventricle; 2.) Earlier endocardial activation than epicardial activation in left ventricular free wall; and 3.) Earlier activation in the apex than in the base of both ventricles. It has been found that a site of earliest activation occurs in the endocardium of the left ventricle along a lower portion of the inter-ventricular septum (i.e. near the apex) where it joins with the anterior wall of the heart. It would be desirable to pace at or near this site of earliest activation.

### BRIEF DESCRIPTION OF THE DRAWINGS

The following drawings are illustrative of particular embodiments of the invention and therefore do not limit its scope, but are presented to assist in providing a proper understanding of the invention. The drawings are not to scale (unless so stated) and are intended for use in conjunction with the explanations in the following detailed description. The present invention will hereinafter be described in conjunction with the appended drawings, wherein like numerals denote like elements, and:

FIG. 1 is a schematic section through a heart wherein a pacing lead according to one embodiment of the present invention is implanted;

FIG. 2 is an enlarged view of a portion of FIG. 1;

FIG. 3 is a schematic section through a portion of a heart wherein a pacing lead according to an alternate embodiment of the present invention is implanted; and

FIG. 4 is a schematic section through a portion of a heart wherein a delivery system according to an embodiment of the present invention is employed.

### DETAILED DESCRIPTION

The following description is exemplary in nature and is not intended to limit the scope, applicability, or configuration of the invention in any way. Rather, the following description provides a practical illustration for implementing exemplary embodiments of the invention.

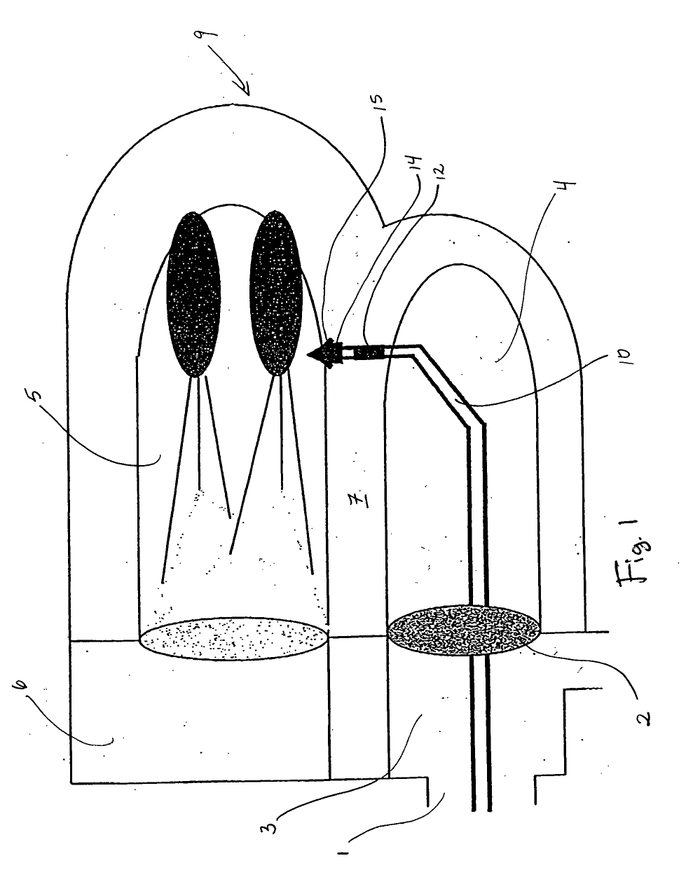

FIG. 1 is a schematic section through a heart wherein a distal portion of a pacing lead 10 according to one embodiment of the present invention is implanted. FIG. 1 illustrates the distal portion of lead 10 extending through a superior vena cava 1, a right atrium 3 and a mitral valve 2 into a right ventricle 4; lead 10 includes an anode electrode 12 and a cathode electrode 14 which are shown implanted within an interventricular septal wall 7 in proximity to a left ventricular apex 9. FIG. 1 further illustrates a tine-like structure 15 terminating a distal end of lead 10, which is within a left ventricle 5. It should be noted that lead 10 may be passed into the right heart via a standard transvenous route which may accessed by cephalic cut-down or subclavian stick; furthermore materials forming lead 10 and an arrangement of conductors, insulation and connector components may all conform to that of standard pacing leads. Tine-like structure 15, according to one embodiment, is formed of a resilient material allowing structure 15 to collapse as the distal portion of lead 10 is inserted through wall 7.

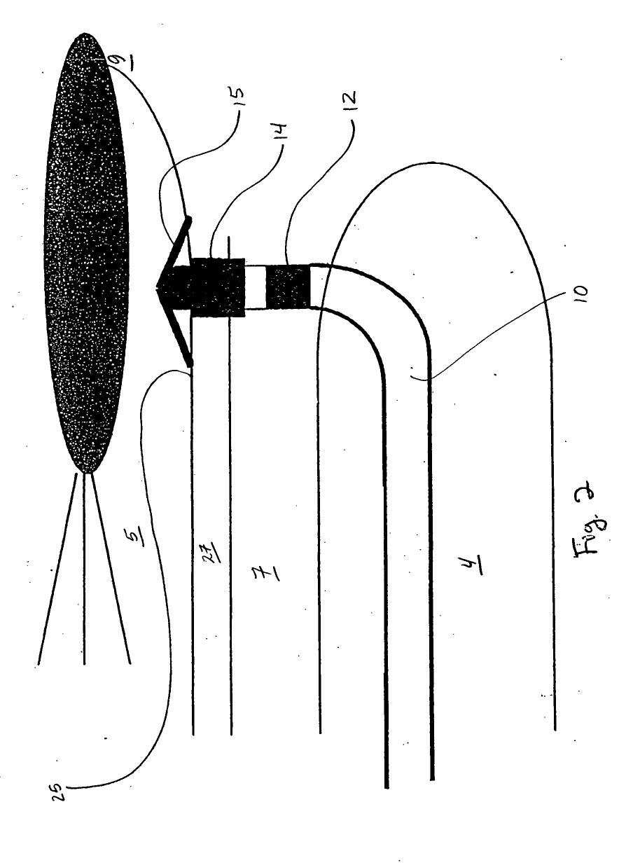

FIG. 2 is an enlarged view of a portion of FIG. 1 showing more specifically an implant site of cathode electrode 14 within a left ventricular endocardial layer 27 of septal wall 7. According to one embodiment of the present invention, the distal portion of lead 10 is inserted through septal wall 7 and then retracted to position electrodes 12 and 14, as illustrated, by means of feeling a resistance of structure 15 against a surface 25 of left ventricular endocardial layer 27; in this way structure 15 can serve as a depth gauge to assure that cathode electrode 14 is positioned for left ventricular endocardial pacing and sensing. It should be noted that alternative geometries of tine-like structures performing a similar function to structure 15, for example a hook geometry, can be incorporated into alternate embodiments of the present invention.

As is further illustrated in FIG. 2, anode electrode 12 is spaced proximally from cathode electrode 14 so that when cathode 14 is positioned in left ventricular endocardial layer 27 anode 12 is positioned within a more central portion of septal wall 7. A thickness of septal wall 7, in proximity to left ventricular apex 9, may be between approximately 1.5 and approximately 2 cm, so that, according to some exemplary embodiments, a spacing between electrodes 14 and 12 is between approximately 5 mm and approximately 12 mm; state of the art electrode features including surface areas, macro and micro, and surface structure and treatments may be incorporated into some embodiments of the present invention. It should be noted that another embodiment of the present invention includes only electrode 14 and stimulation is unipolar, wherein a cardiac rhythm management device (not shown), to which lead 10 is coupled, serves as an indifferent electrode (—such devices and couplings are well known to those skilled in the art).

According to one embodiment of the present invention, tine-like structure 15 is formed of a material adapted to dissolve in the blood soon after lead placement to reduce a risk for thrombus formation about structure 15. Examples of such materials include those taught in lines 10-24 of column 4 of U.S. Pat. No. 6,173,206, which are incorporated by reference herein. FIG. 2 further illustrates cathode electrode 14 sized to serve as an anti-retraction feature, that is electrode 14 is oversized or includes an outer surface protruding radially from an adjacent portion of lead 10 just proximal to electrode 14.

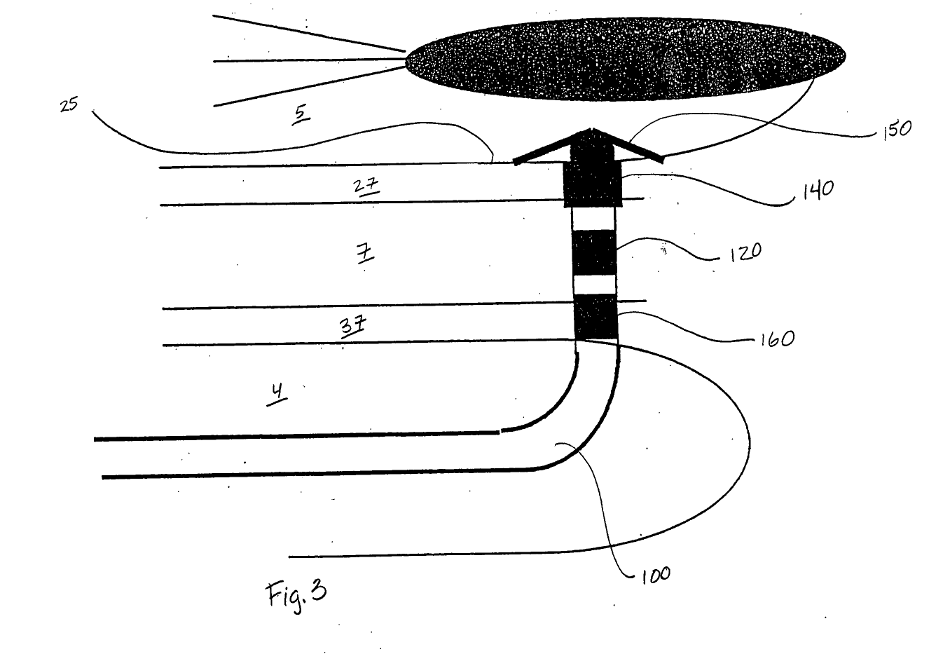

FIG. 3 is a schematic section through a portion of a heart wherein a distal portion of a pacing lead 100 according to an alternate embodiment of the present invention is implanted; lead 100 extends into right ventricle 4 via a path very similar to that illustrated in FIG. 1 for lead 10. FIG. 3 illustrates lead 100 including a first electrode 140, a second electrode 120 and a third electrode 160. According to one embodiment first electrode 140 and third electrode 160 are each cathodes and second electrode 120 is an anode such that two bipolar pairs are formed for pacing and sensing, wherein first electrode 140 and second electrode 120 form a first bipolar pair for left ventricular pacing and sensing and third electrode 160 and second electrode 120 form a bipolar pair for right ventricular pacing and sensing. According to another embodiment, second electrode 120 is not included; in this case first electrode 140 and third electrode 160 are either operated in a unipolar mode or are adapted to alternate between polarities for bipolar operation such that, in one point in time, first electrode 140 is a cathode and third electrode 160 an anode for left ventricular pacing and sensing while, at another point in time, third electrode 160 is the cathode and first electrode 140 is the anode for right ventricular pacing and sensing.

According to some embodiments of the present invention, a pacing interval that appropriately times pacing pulses to right ventricular endocardium 37, via electrode 160, and left ventricular endocardium 27, via electrode 140, is programmed into a cardiac rhythm management device (not shown) to which lead 100 is coupled (-such devices and couplings are well known to those skilled in the art); preferably the interval is in sync with an innate electro-mechanical coupling between the electrode stimulation sites. Such an interval may be between approximately 0.5 milliseconds and approximately 100 milliseconds. Typically, in normal hearts, the natural conduction system activates the left ventricular endocardium prior to the right ventricular endocardium, so that according to one embodiment of the present invention, a pacing interval is set in which left ventricular pacing occurs prior to right ventricular pacing. According to some embodiments of the present invention, biphasic stimulation is incorporated, that is, a polarity for right ventricular pacing is the opposite of that for left ventricular pacing.

FIG. 3 further illustrates lead 100 including a tine-like structure 150; according to one embodiment, structure 150 functions in a manner similar to structure 15 of lead 10 as previously described in conjunction with FIG. 2. First electrode 140 is positioned with respect to structure 150, and second electrode 120 is positioned with respect to first electrode 140, and third electrode 160 is positioned with respect to second electrode 120, so that when lead 100 is implanted as illustrated, with structure 150 positioned in left ventricle 5, adjacent to endocardial surface 25, first electrode 140 is located within left ventricular endocardium 27, second electrode 120 is located within a more central portion of septal wall 7 and third electrode 160 is located within a right ventricular endocardium 37. It should be noted that the scope of the present invention allows for spacings between electrodes (i.e. 12 and 14, 140 and 120, 120 and 160 and 140 and 160) that are not constrained to keep electrodes 14, 140 and 160 completely embedded in endocardial surfaces (i.e. 27 and 37), that is, portions of the cathode surfaces may protrude from the endocardial surfaces into the ventricles or may extend into a more central portion of the septal wall.

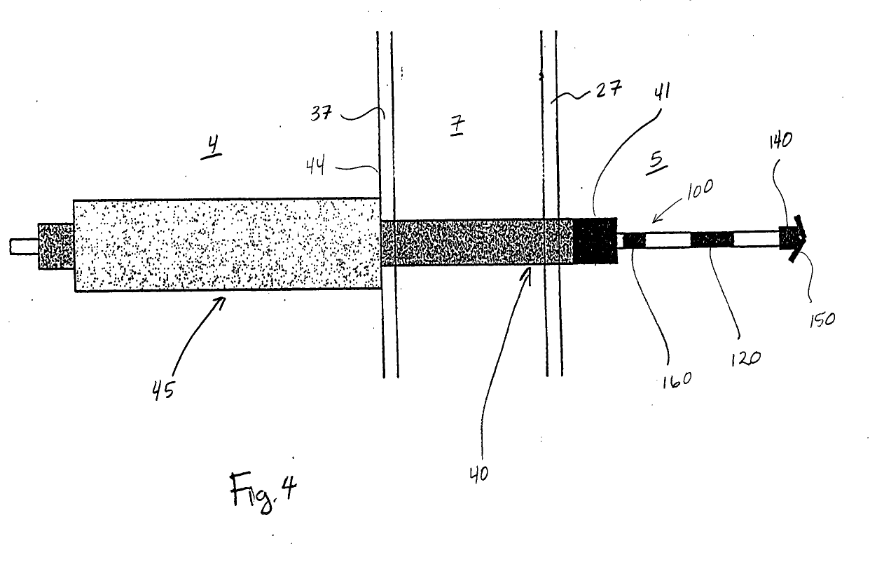

FIG. 4 is a schematic section through a portion of a heart wherein a delivery system according to an embodiment of the present invention is employed. FIG. 4 illustrates a distal portion of the delivery system, which includes a guiding catheter 45, a septal puncture needle 40 slideably received within the guiding catheter 45, and lead 100 slideably received within puncture needle 40. According to the illustrated embodiment, catheter 45 has been positioned against a surface 44 of right ventricular endocardium 37 so that needle 40, passing through catheter 45 may puncture through septal wall 7; guiding catheter 45 may be of a type of guiding catheter well known to those skilled in the art, which is constructed having a shape enabling positioning for a selected puncture site and a stiffness sufficient to provide backup support for puncturing. It may be determined via arterial blood backflow, from left ventricle 5 through needle 40, when needle 40 has punctured through wall 7; once the passageway is established by needle 40, lead 100 is passed through as illustrated. According to other embodiments of the present invention, an alternate method for passing lead 100 through wall includes first piercing through wall 7 with a tool to make a bore and then removing the tool to pass lead 100 through the bore. According to yet another embodiment, tine-like structure 150 includes a piercing tip so that lead 100, reinforced by an internal stiffening stylet may pierce through wall 7 without need for an independent piercing tool.

FIG. 4 further illustrates an electrode 41 coupled to needle 40 in proximity to a distal end of needle 40 which may be used to sense and/or pace as needle 40 passes through septal wall 7. Once lead 100 has been passed through wall 7, as illustrated, needle 40 is pulled back out from wall 7 so that lead 100 may be retracted to position electrodes 140, 120, and 160 within wall, as illustrated in FIG. 3.

Although embodiments of the present invention have been described herein in the context of cardiac pacing, it should be appreciated that embodiments of the present invention may be used for electrical stimulation of any body including a septum wherein it would be desirable to enter the septum from one side and pass through the septum to another side in order to position an electrode at or near that other side. Furthermore it may be appreciated that various modifications and changes can be made to the various embodiments described herein without departing from the scope of the invention as set forth in the appended claims.

1. A method of securely displaying visual data comprising the steps of:
generating a private key and a corresponding public key for a display apparatus;
securely storing the private key within the display apparatus;
communicating the public key from the display apparatus to an encryption apparatus;
encrypting the visual data at the encryption apparatus using the public key, whereby encrypted visual data is formed;
transporting the encrypted visual data from the encryption apparatus to the display apparatus;
decrypting the encrypted visual data within the display apparatus such that an electronic version of the visual data is maintained within circuit elements that are substantially inaccessible; and
displaying the visual data as a visual image.

2. The method of claim 1 wherein the circuit elements comprise integrated circuit elements.

3. The method of claim 2 wherein the integrated circuit elements comprise a display circuit and a diffractive light valve, the diffractive light valve displaying the visual image.

4. The method of claim 3 wherein the diffractive light valve comprises a diffractive light valve.

5. The method of claim 4 wherein the integrated circuit elements comprise portions of a single integrated circuit.

6. The method of claim 4:
wherein the integrated circuit elements comprise individual integrated circuits; and
further comprising the steps of encoding and decoding the visual data in order to transfer the visual data between the individual integrated circuits.

7. The method of claim 4 wherein the display circuit comprises a driver circuit for driving the diffractive light valve.

8. The method of claim 4 wherein the step of displaying the visual data comprises scanning a line image over a display screen such that the visual image has low persistence.

9. The method of claim 4 wherein the integrated circuit elements comprise a decryption circuit.

10. The method of claim 4 wherein the step of transporting the encrypted visual data comprises electronic transmission.

11. The method of claim 10 wherein the electronic transmission is selected from the group consisting of satellite transmission, optical fiber transmission, and internet transmission.

12. The method of claim 4 wherein the step of transporting the encrypted visual data comprises recording the encrypted visual data on a storage medium and physically transporting the storage medium.

13. The method of claim 12 wherein the storage medium comprises a standard storage medium.

14. The method of claim 12 wherein the storage medium comprises a non-standard storage medium.

15. (canceled)

16. The method of claim 1 wherein the step of generating the private key and the corresponding public key takes place within the display apparatus.

17. The method of claim 1
wherein the step of generating the private key and the corresponding public key takes place outside of the display apparatus; and
further comprising the step of inputting the private key to the display apparatus in such a manner that human access to the private key is substantially unavailable.

18. (canceled)

19. A system for securely transmitting and displaying visual data comprising:
an encryption apparatus for encrypting the visual data, whereby encrypted visual data is formed;
means for transporting the encrypted visual data from the encryption apparatus to a display facility; and
a display apparatus located at the display facility that receives the encrypted visual data, the display apparatus decrypting the encrypted visual data such that an electronic version of the visual data is maintained within circuit elements that are substantially inaccessible, the display apparatus displaying the visual data as a visual images,
wherein the encryption apparatus uses a public key for encrypting the visual data, and
wherein the display apparatus uses a private key for decrypting the visual data, the private key residing within the display apparatus.

20. The system of claim 19 wherein the circuit elements comprise integrated circuit elements.

21. The system of claim 20 wherein the integrated circuit elements comprise a display circuit and further wherein the display circuit comprises a diffractive light valve for displaying the visual image.

22. The system of claim 21 wherein the light valve comprises a grating light valve.

23. The system of claim 22 wherein the integrated circuit elements comprise portions of a single integrated circuit.

24. The system of claim 22 wherein the integrated circuit elements comprise individual integrated circuits and further wherein the integrated circuit elements encode and decode the visual data to transfer the visual data between the individual integrated circuits.

25. The system of claim 22 wherein the display apparatus includes a scanning device for scanning a linear image over a display screen such that the visual image has low persistence.

26. The system of claim 22 wherein the means for transporting the encrypted visual data includes means for electronic transmission.

27. The system of claim 26 wherein the means for electronic transmission is selected from the group consisting of satellite transmission, optical fiber transmission, and internet transmission.

28. The system of claim 22 wherein the means for transporting the encrypted visual data includes a storage medium, the storage medium holding the encrypted visual data during transport of the storage medium.

29. The system of claim 28 wherein the storage medium comprises a standard storage medium.

30. The system of claim 28 wherein the storage medium comprises a non-standard storage medium.

31. (canceled)

32. The system of claim 19 wherein the display apparatus generates the public key and the private key.

33. The system of claim 19 wherein the public key and the private key have been generated outside of the display apparatus and further wherein the private key has been generated an input to the display apparatus in such a manner that human access to the private key is substantially unavailable.

34. (canceled)

35. A display apparatus for displaying encrypted visual data comprising circuit elements that are substantially inaccessible, the circuit elements comprising a decryption circuit for decrypting the encrypted visual data, whereby visual data is formed, the circuit elements comprising a display circuit for displaying the visual data as a visual image, such that an electronic version of the visual data is maintained within the circuit elements, wherein the display apparatus uses a private key for decrypting the encrypted visual data, wherein the private key resides within the display apparatus, and wherein the encrypted visual data was previously generated using a public key corresponding to the private key.

36. The display apparatus of claim 35 wherein the display circuit comprises a diffractive light valve for displaying the visual image.

37. The display apparatus of claim 36 wherein the diffractive light valve is a grating light valve.

38. A display apparatus for displaying encrypted visual data comprising:
a decryption circuit for decrypting the encrypted visual data, whereby the visual data is formed; and
a diffractive light valve for displaying the visual data as a visual image,
wherein the display apparatus uses a private key for decrypting the encrypted visual data,
wherein the private key resides within the display apparatus, and
wherein the encrypted visual data was previously generated using a public key corresponding to the private key.
